(19) United States
(12) Patent Application Publication
(10) Pub. No.: US 2021/0293711 A1
(43) Pub. Date: Sep. 23, 2021

(54) BIOPOLYMER ANALYSIS METHOD AND BIOPOLYMER ANALYSIS DEVICE

(71) Applicant: Hitachi High-Tech Corporation, Tokyo (JP)

(72) Inventors: Satoshi Takahashi, Tokyo (JP); Michiru Fujioka, Tokyo (JP); Mikihiro Kobayashi, Tokyo (JP); Isao Haraura, Tokyo (JP); Takeshi Ooura, Tokyo (JP); Motohiro Yamazaki, Tokyo (JP)

(73) Assignee: Hitachi High-Tech Corporation, Tokyo (JP)

(21) Appl. No.: 17/263,602

(22) PCT Filed: Aug. 2, 2018
(86) PCT No.: PCT/JP2018/029093

§ 371 Date: Jan. 27, 2021

(51) Int. Cl. G01N 21/64; C12Q 1/6869; G01N 27/447

(52) U.S. Cl. CPC G01N 21/6428; C12Q 1/6869; G01N 21/6402; G01N 2201/06113; G01N 27/44726; G01N 2021/6439; G01N 27/44791

(57) ABSTRACT

There is a phenomenon where a noise in a spike shape caused by, for example, impurities and a noise peak having a spectrum different from a wavelength spectrum of a labeled fluorescent substance are detected during a capillary electrophoresis. Therefore, the disclosure provides a technique to identify an intensity of the labeled fluorescent substance itself without an effect by a noise fluorescence peak caused by impurities. In the disclosure, a fluorescence intensity property (a fluorescence profile of a noise) common to the noise peaks is set, the noise peak is handled as a fluorescent substance different from the labeled fluorescent substance, and the fluorescent substance and the noise are separated by color converting with the labeled fluorescent substance+the noise fluorescent substance (see FIG. 5).

### TECHNICAL FIELD

The disclosure relates to a biopolymer analysis method and a biopolymer analyzer.

### BACKGROUND ART

As a method for determining a base sequence of a DNA labeled with a fluorescent substance, for example, there is a well-known dideoxy method by Sanger et al. In this dideoxy method, the DNA to be analyzed is first introduced into a vector and amplified, and then is denatured into a single-stranded template DNA. Then, a primer DNA is bound to this template DNA, and a complementary strand synthesis starting from the primer DNA. In this respect, besides four types of deoxynucleotide triphosphates, a specific one type of dideoxynucleotide triphosphate that serves as a terminator is added. When this dideoxynucleotide triphosphate is incorporated, the complementary strand synthesis stops, and thus, various lengths of DNA fragments terminated with specific bases are obtained. Dideoxynucleotide triphosphates for four types of bases, adenine (A), cytosine (C), guanine (G), and thymine (T), namely, ddATP, ddCTP, ddGTP, and ddTTP are used, the respective complementary strand synthesis reactions described above are performed to obtain various lengths of DNA fragments having respective terminal bases of A, C, G, and T, these DNA fragments are separated by molecular weight separation, and the base species are read in the order according to the molecular weights, thereby ensuring an analysis of a base sequence.

The molecular weight separation is performed by electrophoresis using, for example, a polyacrylamide gel. In recent years, the main method is to fill a gel or a polymer that allows the molecular weight separation in a capillary and performs the electrophoresis. A base sequence determination device for DNA (DNA sequencer) using this capillary electrophoresis method currently is the most widely spread DNA sequencer that accommodates a continuous automatic analysis to ensure simultaneously analyzing multiple of samples at high speed.

The principle of determining the base species of the detected fragments is that the above-described fragments are preliminarily labeled with four types of fluorescent substances different by each terminal base species and irradiated with an excitation light at a specific detecting position, and the base species are determined from differences between the generated fluorescence spectra. An apparatus based on this principle can be widely utilized in analyzing fluorescently labeled biologically-relevant substances, besides a usage as a DNA sequencer.

While there can be various combinations of fluorescent substances used as labels, in the case of, for example, four types of fluorescent substances with different properties, such as blue, green, yellow, and red are selected. The fluorescent substances having respective fluorescence maximum wavelengths of 528 nm, 549 nm, 575 nm, and 602 nm, which are shifted from one another, are used. A fluorescent substance type can be identified or a mixture states of fluorescent substance types can be identified from the difference of these fluorescence maximum wavelengths and the difference of fluorescence spectra, and thus, the terminal base species can be determined. For a method for computing a fluorescent substance type from the detected fluorescence spectrum, for example, a well-known method as described in Patent Literature 1 is used.

While the fluorescent substance types used as labels are usually four types in determining a base sequence, there also is a measurement that uses five types or more to label each fragment type with a different fluorescent substance, and measure a molecular weight separation pattern of DNA. Use of five types or more allows identification of the fluorescent substance type from the fluorescence spectrum and the like from the detected fluorescent substance, and determine a fragment type and its length.

A measurement device has a function to measure fluorescence intensities in different wavelength bands of at least equal to or more than the number of the fluorescent substance types. Fluorescent substances have fluorescence spectra different from one another, and a fluorescence intensity ratio for each of a plurality of the wavelength bands based on a spectrum property differs by each fluorescent substance type. Therefore, from the fluorescence intensities of the detected plurality of wavelength bands and the fluorescence intensity ratio for each fluorescent substance type, an intensity (amount) for each fluorescent substance type is converted using a matrix calculation. Since an amount of the fluorescent substance type is an amount of the base species, an amount for each base can be computed, and a temporal change by each base caused by electrophoresis can be obtained.

For example, Patent Literature 2 describes a capillary electrophoretic apparatus as described above. Generally, in a capillary electrophoretic apparatus, a sample containing DNA as a measurement target is injected in a separation medium, such as polyacrylamide, in a quartz capillary, and a voltage is applied to both ends of the capillary. The samples containing the DNAs in a sample moves within the capillary and is separated according to, for example, sizes of molecular weights, and DNA bands are generated in the capillary. Since each DNA band contains fluorochromes as described above, it emits fluorescence by an irradiation with a laser light, a LED light, or the like. Reading this fluorescence emission with a fluorescence measuring means ensures determining a sequence of the DNA. Separation and analysis of protein can be similarly performed to examine a composition of the protein. A light irradiation method onto a sample in the capillary electrophoretic apparatus is as follows. That is, a capillary at one end or capillaries at both ends of a capillary array configured of a plurality of capillaries arranged on a flat substrate are irradiated with a laser light, and the laser light sequentially propagates to adjacent capillaries to traverse the capillary array, and thus, the sample being electrophoresed through all the capillaries is irradiated. In addition, a fluorescence detection method is as follows. That is, an image of a laser light irradiation unit on the capillary array is formed on a two-dimensional CCD through a condensing lens, a transmissive diffraction grating, and an imaging lens. This detects intensities of the fluorescence lights from the plurality of fluorescent substances in a plurality of wavelength bands (for example, dividing a wavelength band from 500 nm to 700 nm into twenty divisions of 10 nm each).

### CITATION LIST

### Patent Literature

Patent Literature 1: Japanese Unexamined Patent Application Publication No. 2011-30502

Patent Literature 2: Japanese Unexamined Patent Application Publication No. 2004-144479

### SUMMARY OF INVENTION

### Technical Problem

As disclosed in Patent Literature 1, the fluorescent substance as a detection target is a fluorescent substance used as a label (a labeled fluorescent substance). That is, by the above-described the matrix calculation, the conversion is to determine which fluorescent substance type (base species) the detected fluorescence intensity is derived from or how much the mixing ratio of fluorescent substance types (base species) is.

However, there is a case where a component other than target (detection target) is electrophoresed and detected in the electrophoresis. For example, it is a case where impurities, dust, and the like contained in the electrophoresis sample are electrophoresed and passed through a detection area of the capillary. A noise fluorescence peak caused by these impurities may overlaid on a peak signal of the native labeled fluorescent substance or may be detected by itself. In such a case, it is concerned that the noise fluorescence peak affects in converting into a fluorescent substance type and determining a base species, for example, by being falsely determined as any one of the labeled fluorescent substances or a combination of the plurality of labeled fluorescent substances.

Since this noise fluorescence signal is different from the fluorescence spectrum of the labeled fluorescent substance as a target, the conversion is not properly performed with a matrix conversion that converts an ordinary fluorescence spectral intensity into a fluorescent substance type intensity. The noise fluorescence signal is overlaid by the fluorescent substance type intensity and is calculated to an inaccurate intensity, thereby causing a problem that the noise fluorescence signal affects the determination of fragment type and base species.

The disclosure has been made in consideration of such a circumstance, and provides a technique to identify an intensity of a labeled fluorescent substance itself without an effect of a noise fluorescence peak caused by impurities.

### Solution to Problem

As a result of analyzing an electrophoresis peak other than a fluorescent substance used as a label, the inventors have found that it has a different spectrum from a fluorescence spectrum of the fluorescent substance used as a label and there is a communality between spectra of a plurality of impurity fluorescence peaks.

Therefore, based on the finding by the inventors, the disclosure handles the noise fluorescence as an electrophoresed fluorescent substance, and determines a fluorescence intensity ratio for each of a plurality of wavelength bands of the noise fluorescence, similarly to other labeled fluorescent substances. In a matrix conversion for converting a fluorescence spectral intensity into a fluorescent substance type intensity, a calculation is performed as a matrix of a labeled fluorescent substance (Q type) and a noise fluorescent substance (R type) to compute a concentration of the labeled fluorescent substance. This ensures identifying the noise fluorescence peak as a noise to enable it to be removed from the peak of the labeled fluorescent substance.

Further features pertaining to the disclosure will be apparent from the description herein and the attached drawings. Aspects of the disclosure are achieved and implemented by elements, combinations of various elements, and modes of the following detailed description and attached claims.

The description herein is merely a typical example, and it should be understood that the claims or the application examples are not intended to limit in any sense.

### Advantageous Effects of Invention

With the disclosure, even when a noise fluorescence peak caused by impurities is detected in electrophoresis data (electropherogram), an intensity of a labeled fluorescent substance itself can be computed without an effect of the noise fluorescence peak, thereby ensuring accurately identifying and detecting a biologically-relevant element, such as a base species.

### BRIEF DESCRIPTION OF DRAWINGS

FIG. 1 is a drawing illustrating an exemplary schematic configuration of a capillary electrophoretic apparatus 100 according to the embodiment.

FIG. 2 is a drawing illustrating an exemplary schematic internal configuration (light detection system) of a detection mechanism unit 37 as a component of the capillary electrophoretic apparatus 100 according to the embodiment.

FIG. 3 is a flowchart for describing an electrophoresis data analyzing process executed by a data processing unit 101 based on an analysis method 1.

FIG. 4 is a flowchart for describing an electrophoresis data analyzing process executed by the data processing unit 101 based on an analysis method 2.

FIG. 5 is a drawing illustrating an effect of a noise fluorescence removal according to Example 1.

FIG. 6 is a drawing illustrating an effect of a noise fluorescence removal according to Example 2.

FIG. 7 is a drawing illustrating fluorescence spectral profiles: x (q, p) of labeled fluorescent substances used in Example 3 and a noise fluorescence profile: y (r, p) (q=0, 1, 2, 3, r=0, p=0, 1, 2, . . . , 19).

FIG. 8 is a drawing illustrating an exemplary electropherogram s (p, t) during electrophoresis measured.

FIG. 9 is a drawing illustrating a part of an intensity waveform: n (r, t) of a noise fluorescent substance analyzed by the least squares method (a signal intensity 901 of a noise fluorescence

FIG. 10 is a drawing illustrating a result of a noise peak removal by an operation (fluorescence intensity waveforms f (q, t) from respective labeled fluorescent substances: intensity waveforms 1001 to 1004 of fluorescent substances 1 to 4).

FIG. 11 is a drawing illustrating fluorescence intensity waveforms from labeled fluorescent substances (intensity waveforms 1101 to 1104 of the fluorescent substances 1 to 4) calculated without setting the noise fluorescent substance as a Comparative Example.

FIG. 12 is a drawing illustrating four types of labeled fluorescent substance profiles (profiles 1201 to 1204 of the fluorescent substances 1 to 4) and two types of noise fluorescent substance profiles (profiles 1205 and 1206 of the noise fluorescences 1 and 2).

FIG. 13 is a drawing illustrating fluorescence spectral profiles: x (q, p) (q=0, 1, 2, 3, 4, p=0, 1, 2, . . . , 19) of labeled fluorescent substances used in Example 4.

FIG. 14 is a drawing illustrating a noise fluorescence profiles: y (r, p) (r=0, 1, p=0, 1, 2, . . . , 19) used in Example 4.

FIG. 15 is a drawing illustrating an exemplary electropherogram s (p, t) during electrophoresis measured.

FIG. 16 is a drawing illustrating parts of intensity waveforms: n (r, t) of the noise fluorescent substances calculated in accordance with the analysis method 1.

FIG. 17 is a drawing illustrating a result obtained by an operation according to the analysis method 1 (fluorescence intensity waveforms: f (q, t) from the labeled fluorescent substances during electrophoresis).

FIG. 18 is a drawing illustrating fluorescence intensity waveforms from the labeled fluorescent substances calculated without setting fluorescence profiles y (r, p) of the noise fluorescent substances as a Comparative Example.

### DESCRIPTION OF EMBODIMENTS

The following describes the embodiment and examples with reference to the attached drawings. In the attached drawings, elements that share functionality may be indicated with the same number. Note that while the attached drawings illustrate specific embodiment and examples based on the principle of the disclosure, they are for understanding the disclosure, and never used for interpreting the disclosure in a limited way.

While in the embodiment, the explanation of the disclosure is made sufficiently in detail for a person skilled in the art to execute the disclosure, other implementations and configurations are possible, and it is necessary to be understood that changes in configurations and structures and replacements of various components are possible without departing from the scope and spirit of the technical idea of the disclosure. Accordingly, the following description may not be interpreted in a limited way.

Furthermore, in the embodiment, the function of a data processing unit described later may be implemented in software that operates on a general-purpose computer or may be implemented in dedicated hardware or a combination of software and hardware.

The embodiment relates to an analysis technique of a biologically-relevant element (a biopolymer), such as DNA and protein using a fluorescent substance as a label, and relates, for example, to a method for determining a base sequence of DNA and an apparatus thereof or a method for measuring a molecular weight separation pattern of DNA and an apparatus thereof.

The embodiment presets a profile of a fluorescent substance (a non-labeled fluorescent substance) other than a labeled fluorescent substance (for example, a profile of a noise) as well as a profile of the labeled fluorescent substance, obtains a temporal change (a waveform) of a fluorescence intensity of the non-labeled fluorescent substance by an operation (see formula (1) and the like described later) using the profile of the non-labeled fluorescent substance, and removes the noise from detected electropherogram signals. In addition, the embodiment directly obtains a fluorescence intensity from the labeled fluorescent substance during electrophoresis by an operation from the formula (1) described below.

### <Exemplary Configuration of Capillary Electrophoretic Apparatus>

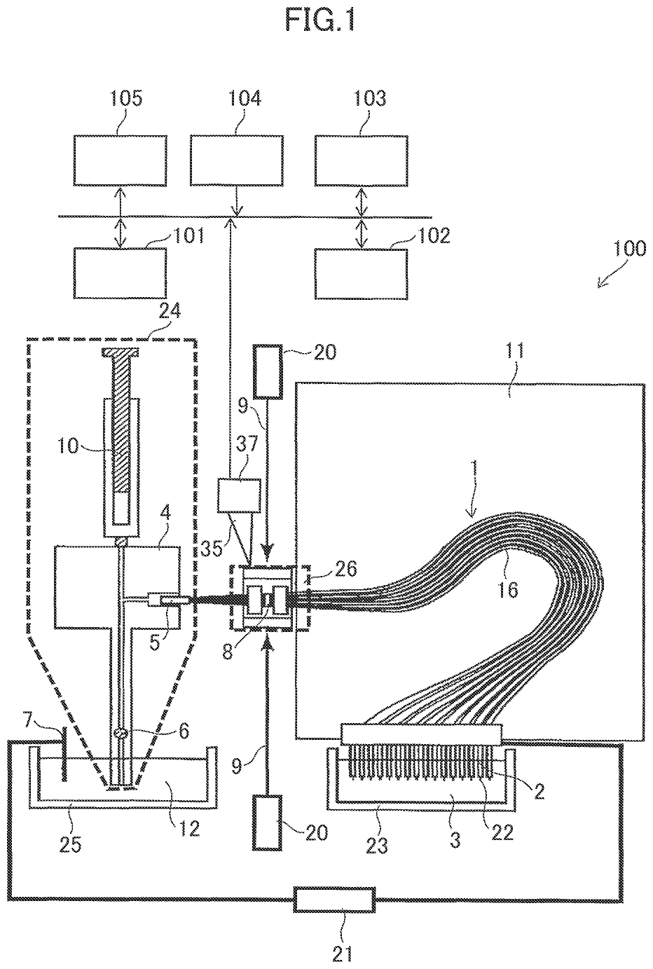

FIG. 1 is a drawing illustrating an exemplary schematic configuration of a capillary electrophoretic apparatus 100 according to the embodiment. The capillary electrophoretic apparatus 100 is a biopolymer analyzer and includes, for example, a multiple capillary array 1, a first buffer container 23, a gel block 4, a second buffer container 25, a syringe 10, a detector 26, a light source 20, a detection mechanism unit 37, an oven 11, a high-voltage power supply 21, a data processing unit (a processor) 101, a memory 102, a storage device 103, an input device (for example, a computer mouse, a keyboard, various kinds of switches, and a touch panel) 104, and an output device (for example, a display device and a speaker that emits an alarm and the like) 105. The multiple capillary array 1 is formed of capillaries that contain a separation medium for separating a sample. The first buffer container 23 holds a buffer liquid 3 in which negative electrodes 2 of the multiple capillary array and sample introduction portions 22 are immersed. The gel block 4 includes a valve 6. The second buffer container 25 holds a buffer liquid 12 in which the gel block 4 and an earth electrode 7 are immersed. The syringe 10 is for injecting a gel as an electrophoresis medium into the capillary array. The detector 26 is for obtaining information depending on the sample. The light source 20 irradiates a light irradiation position 8 with a laser light 9 for exciting fluorescent substances within the electrophoresed sample. The detection mechanism unit 37 obtains fluoresce generated from the sample. The oven 11 adjusts temperature of the capillary array 1. The high-voltage power supply 21 applies a voltage to the separation medium. The data processing unit 101 executes various kinds of processes. The memory 102 stores a profile of each labeled fluorescent substance (synonymous with a fluorescence spectral profile) and each noise fluorescence profile described below. The storage device 103 stores detection data and arithmetic operation results of the past. The input device 104 receives operator inputs, for example, instructions and various kinds of data. The output device 105 outputs detection (measurement) results, arithmetic operation results, determination results, and the like.

The multiple capillary array 1 is employed and configured of a plurality (for example, 96, 24, 16, 12, and 8) of capillaries 16 made of quartz as tubular members arranged in an aligned manner on a plane in the detector 26 that includes the light irradiation position (the position irradiated with the laser light 9) 8. While each of the capillaries 16 is coated with polyimide or the like, the coating is removed in the light irradiation position 8 to allow a light irradiation. The multiple capillary array 1 is filled with an examination sample containing a sample, such as DNA molecules, and a polymer aqueous solution as a separation medium for separating the DNA molecules in the examination sample. The multiple capillary array 1 has one side ends at which the sample introduction portions 22 that can introduce the sample into the capillaries 16 are formed and the negative electrodes 2 that can apply a negative voltage are disposed. The other end has a gel block coupling portion 5 coupled to the gel block 4, and the separation medium (for example, the polymer aqueous solution having a molecular sieve effect) is injected from the gel block 4 into the capillary array 1. The detector 26 is disposed between the sample introduction portion 22 and the gel block coupling portion 5.

A flow medium injection mechanism 24 that injects the polymer aqueous solution as an electrophoresis separation medium into the capillaries 16 includes the gel block 4, the syringe 10, and the valve 6. When the polymer aqueous solution as the electrophoresis medium is filled into each of the capillaries 16, for example, a control unit (not illustrated) closes the valve 6 and pushed the syringe 10 in to inject the polymer aqueous solution within the syringe 10 into the capillaries.

The capillary array 1, the gel block 4, the buffer liquid 3, the negative electrode 2, the buffer 12 in an earth electrode side, the earth electrode 7, and the high-voltage power supply 21 configure a voltage application mechanism for electrophoresing the examination sample within the separation medium (the polymer aqueous solution). When it is electrophoresed, the negative electrode 2 is immersed in the buffer liquid 3, and the control unit (not illustrated) opens the valve 6. This forms a conduction path made of the negative electrode 2, the buffer liquid 3, the capillary array (more precisely, the polymer aqueous solution in each capillary 16) 1, the gel block (more precisely, the polymer aqueous solution in the gel block 4) 4, the buffer 12 in the earth electrode side, and the earth electrode 7. The high-voltage power supply 21 applies a voltage to this conduction path. When the voltage is applied to the conduction path, the examination sample is electrophoresed within the separation medium (the polymer aqueous solution) and is separated in accordance with properties of its molecular weights and the like.

An optical system of the electrophoretic apparatus 100 is configured of the light source 20, the detector 26 including the light irradiation position 8, and the detection mechanism unit 37 detecting a fluorescent light 35 generated from the detector 26. The light source 20 oscillates the laser light 9 (lights of 488.0 nm and 514.5 nm). Instead of the laser light 9, an LED light monochromatized by, for example, a band-pass filter or a light emitted from another light source capable of fluorescence excitation may be used. In the detector 26, the light irradiation positions 8, which are positions where the laser light 9 transmits through the capillary array 1, are disposed in parallel. The detector 26 is irradiated with the laser light 9 from both directions (up and down directions in FIG. 1) in the arrangement of the capillaries 16 such that the laser light 9 simultaneously penetrates the light irradiation positions 8 of the plurality of capillaries. This laser light 9 excites the examination sample to cause the examination sample to emit fluorescence. The detection mechanism unit 37 including a two-dimensional detector 34 detects this fluorescence, and thus, information depending on the examination sample, such as a DNA molecular sequence, can be obtained.

### <Exemplary Internal Configuration of Detection Mechanism Unit 37>

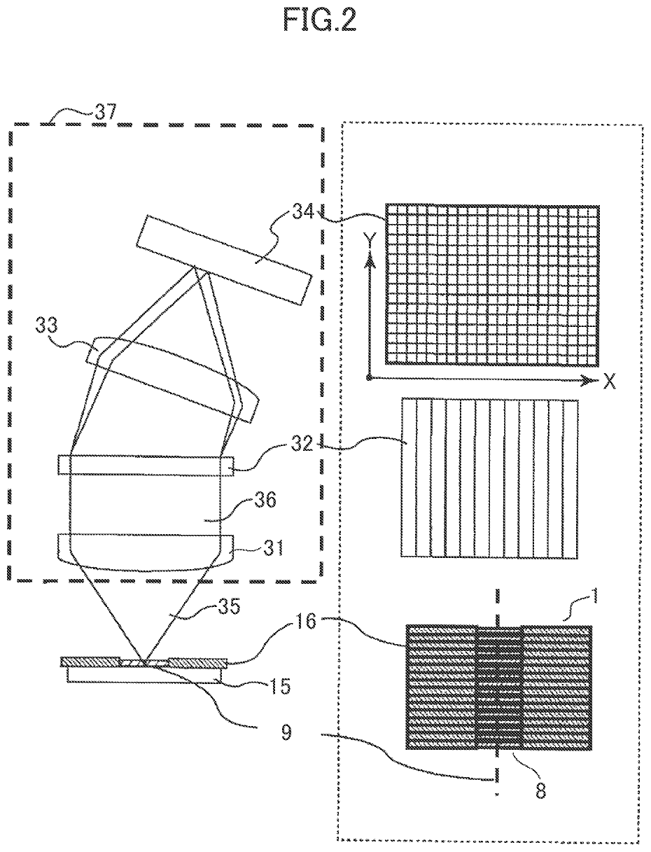

FIG. 2 is a drawing illustrating an exemplary schematic internal configuration (light detection system) of the detection mechanism unit 37 as a component of the capillary electrophoretic apparatus 100 according to the embodiment. FIG. 2 illustrates the detection mechanism unit 37 and the light irradiation position 8.

The detection mechanism unit 37 includes a fluorescence condenser lens 31, a grating 32, a focus lens 33, and the two-dimensional detector 34, such as a CCD camera and a CMOS camera. While it is not illustrated, an optical filter for removing an excitation light may be inserted in the middle of an optical path as necessary. The fluorescent light 35 from the examination sample in the capillaries 16 placed on an array platform 15 is generated by irradiating the light irradiation position 8 with the laser light 9. The fluorescent light 35 becomes a parallel light 36 with the fluorescence condenser lens 31, is dispersed with the grating 32, and forms an image on the two-dimensional detector 34 with the focus lens 33. On the right side in FIG. 2, an exemplary configuration of elements (the capillary array 1, the light irradiation position 8, the grating 32, and the two-dimensional detector 34) relating to the image forming is illustrated. Array images (sixteen in the drawing) of the capillary array 1 are arranged in the Y-axis direction, images are formed by the light emission from each of the capillaries 16 being dispersed in the X-axis direction, and a fluorescence intensity in a wavelength different per pixel in the X-axis direction of the two-dimensional detector is detected. The data processing unit 101 analyzes signals of the detected fluorescence intensities in response, for example, to an instruction input by an operator from the input device 104, to determine a base sequence and the like. The data processing unit 101 also outputs (displays) the signals of fluorescence intensities, the base sequence as the analysis result, and the like on the output device 105 in response, for example, to an instruction input by an operator.

### <Outline of Electrophoresis Data Analysis>

The following describes an outline of an electrophoresis data (electropherogram) analysis according to the embodiment.

The electrophoretic apparatus 100 repeatedly detects signals during electrophoresis at a specified time (the detection may be executed periodically or at every predetermined period using a continuous irradiation with the laser light 9 or the irradiation timing with the laser light 9 and the signal detection timing may be synchronized). Note that the repeated count is t (when one measurement is taken in one second, count=time (second)).

The fluorescence emitted from each labeled fluorescent substance emits a light at a specific intensity ratio by each spectral wavelength according to each fluorescence spectrum. This is dispersed by, for example, grating and prism, and detected with the detector. Based on a combination of the labeled fluorescent substances, a detection wavelength range from a wavelength W1 to a wavelength W2 is set, and the fluorescence within this range is divided into a plurality of wavelength bands and detected. For example, A wavelength region from 520 nm to 700 nm is divided by dividing a sensor surface of the two-dimensional detector 34 into twenty continuous wavelength bands and detected. Thus, when a divided wavelength band number is p (=0, 1, 2, . . . , P−1; P=the number of divisions), a number of a labeled fluorescent substance type is q (=0, 1, 2, . . . , Q−1; Q=the number of fluorescent substance types), and a number of a noise fluorescence r (=0, . . . , R−1; R=the number of set noise fluorescent substance types), respective signal components at a time t is represented as follows.

an electropherogram signal by each divided wavelength band detected: s (p, t)

a fluorescence intensity from a labeled fluorescent substance during electrophoresis: f (q, t)

an intensity of an electrophoresed fluorescent noise: n (r, t)

a background intensity for each divided wavelength band: b (p, t)

a fluorescence profile of a labeled fluorescent substance: x (q, p)

a fluorescence profile of a set noise: y (r, p)

The s (p, t) is an intensity detected (a signal measured) by dividing into the plurality of wavelength bands. The f (q, t) is a fluorescence intensity of each fluorescent substance emitted from an electrophoresed band and the like. The n (r, t) is an intensity of a noise considered to be included in the electrophoresed band. The b (p, t) is a background intensity of each detection wavelength band. The background intensity is an intensity of a signal that serves as a base line, and can be obtained by extracting a signal that fluctuates in a non-pulsive manner in the actually detected s (p, t). The x (q, p) is a fluorescence profile of each labeled fluorescent substance, and is a profile that is obtained by standardizing an intensity detected per detection wavelength band (p) when each labeled fluorescent substance (labeled fluorescent substance type (q)) itself emits a light by each fluorescent substance type. This is uniquely specified when the labeled fluorescent substance is determined, and corresponds to a fluorescence spectrum. The y (r, p) is a profile computed similarly to x (q, p) for the fluorescence regarded as a noise, and set corresponding to a fluorescence spectrum of the noise. For example, this is a profile extracted by analyzing the noise based on accumulated detection data (or assuming what property the noise has). Note that while it is expressed as the “profile of noise” here, it is possible to be expressed as a profile of another fluorescent substance different from the labeled fluorescent substance.

When a matrix representing s (0, t), . . . , s (P−1, t) in P rows and 1 column is S, a matrix representing f (0, t), . . . , f (Q−1, t) in Q row and 1 column is F, a matrix representing n (0, t), . . . , n (R−1, t) in R row and 1 column is N, a matrix representing b (0, t), . . . , b (P−1, t) in P rows and 1 column is B, a matrix representing x (0, 0), . . . , x (Q−1, P−1) in P rows and Q column is X, and a matrix representing y (0, 0), . . . , y (R−1, P−1) in P rows and R column is Y in the above, a formula (1) can be expressed. Note that the matrices S, F, N, B, X, and Y are expressed in italic bold in the formula (1).

$$[ Math . 2 ]$$

$$( 1 )$$

$$S = XF + YN + B$$

$$S = ( \begin{matrix}s ( 0 , t ) \\ s ( 1 , t ) \\ s ( 2 , t ) \\ . \\ . \\ . \\ s ( P - 1 , t )\end{matrix} )$$

$$F = ( \begin{matrix}f ( 0 , t ) \\ f ( 1 , t ) \\ . \\ . \\ . \\ f ( Q - 1 , t )\end{matrix} )$$

$$N = ( \begin{matrix}n ( 0 , t ) \\ . \\ n ( R - 1 , t )\end{matrix} )$$

$$B = ( \begin{matrix}b ( 0 , t ) \\ b ( 1 , t ) \\ b ( 2 , t ) \\ . \\ . \\ . \\ b ( P - 1 , t )\end{matrix} )$$

$$X = ( \begin{matrix}x ( 0 , 0 ) & x ( 1 , 0 ) & … & x ( Q - 1 , 0 ) \\ x ( 0 , 1 ) & x ( 1 , 1 ) & … & x ( Q - 1 , 1 ) \\ x ( 0 , 2 ) & x ( 1 , 2 ) & … & x ( Q - 1 , 2 ) \\ . & . & … & . \\ . & . & … & . \\ . & . & … & . \\ x ( 0 , P - 1 ) & x ( 1 , P - 1 ) & … & x ( Q - 1 , P - 1 )\end{matrix} )$$

$$V = ( \begin{matrix}y ( 0 , 0 ) & … & y ( R - 1 , 0 ) \\ y ( 0 , 1 ) & … & y ( R - 1 , 1 ) \\ y ( 0 , 2 ) & … & y ( R - 1 , 2 ) \\ . & … & . \\ . & … & . \\ . & … & . \\ y ( 0 , P - 1 ) & … & y ( R - 1 , P - 1 )\end{matrix} )$$

For example, when the number of divisions is twenty, the number of labeled fluorescent substances is six, and the number of noise fluorescent substances is two, it can be expressed as a formula (2).

$$[ Math . 2 ]$$

$$( 2 )$$

$$( \begin{matrix}s ( 0 , t ) \\ s ( 1 , t ) \\ s ( 2 , t ) \\ . \\ . \\ . \\ s ( 18 , t ) \\ s ( 19 , t )\end{matrix} ) = ( \begin{matrix}x ( 0 , 0 ) & x ( 1 , 0 ) & … & x ( 5 , 0 ) \\ x ( 0 , 1 ) & x ( 1 , 1 ) & … & x ( 5 , 1 ) \\ x ( 0 , 2 ) & x ( 1 , 2 ) & … & x ( 5 , 2 ) \\ . & . & . & . \\ . & . & . & . \\ . & . & . & . \\ x ( 0 , 18 ) & x ( 1 , 18 ) & … & x ( 5 , 18 ) \\ x ( 0 , 19 ) & x ( 1 , 19 ) & … & x ( 5 , 19 )\end{matrix} ) ( \begin{matrix}f ( 0 , t ) \\ f ( 1 , t ) \\ f ( 2 , t ) \\ f ( 3 , t ) \\ f ( 4 , t ) \\ f ( 5 , t )\end{matrix} ) + ( \begin{matrix}y ( 0 , 0 ) & y ( 1 , 0 ) \\ y ( 0 , 1 ) & y ( 1 , 1 ) \\ y ( 0 , 2 ) & y ( 1 , 2 ) \\ . & . \\ . & . \\ . & . \\ y ( 0 , 18 ) & y ( 1 , 18 ) \\ y ( 0 , 19 ) & y ( 1 , 19 )\end{matrix} ) ( \begin{matrix}n ( 0 , t ) \\ n ( 1 , t )\end{matrix} ) + ( \begin{matrix}b ( 0 , t ) \\ b ( 1 , t ) \\ b ( 2 , t ) \\ . \\ . \\ . \\ b ( 18 , t ) \\ b ( 19 , t )\end{matrix} )$$

In the formula (1), putting the matrix F and the matrix N together and replacing it with a matrix G in (Q+R) rows and 1 column and putting the matrix X and the matrix Y together and replacing it with a matrix Z in P rows and (Q+R) columns ensure an expression as a formula (3). When the number of divisions P is twenty, the number of labeled fluorescent substances Q is six, and the number of noise fluorescent substances R is two, the formula (3) can be expressed as a formula (4). Note that the matrices S, G, B, and Z are expressed in italic bold in the formula (3).

$$[ Math . 3 ]$$

$$( 3 )$$

$$S = ZG + B G = ( \begin{matrix}g ( 0 , t ) \\ g ( 1 , t ) \\ . \\ . \\ . \\ g ( Q + R - 1 , t )\end{matrix} ) = ( \begin{matrix}f ( 0 , t ) \\ f ( 1 , t ) \\ . \\ . \\ . \\ f ( Q - 1 , t ) \\ n ( 0 , t ) \\ . \\ . \\ . \\ n ( R - 1 , t )\end{matrix} ) \\ Z = ( \begin{matrix}z ( 0 , 0 ) & z ( 1 , 0 ) & … & z ( Q + R - 1 , 0 ) \\ z ( 0 , 1 ) & z ( 1 , 1 ) & … & z ( Q + R - 1 , 1 ) \\ z ( 0 , 2 ) & z ( 1 , 2 ) & … & z ( Q + R - 1 , 2 ) \\ . & . & … & . \\ . & . & … & . \\ . & . & … & . \\ z ( 0 , P - 1 ) & z ( 1 , P - 1 ) & … & z ( Q + R - 1 , P - 1 )\end{matrix} ) = \\ ( \begin{matrix}x ( 0 , 0 ) & x ( 1 , 0 ) & … & x ( Q - 1 , 0 ) & y ( 0 , 0 ) & … & y ( R - 1 , 0 ) \\ x ( 0 , 1 ) & x ( 1 , 1 ) & … & x ( Q - 1 , 1 ) & y ( 0 , 1 ) & … & y ( R - 1 , 1 ) \\ x ( 0 , 2 ) & x ( 1 , 2 ) & … & x ( Q - 1 , 2 ) & y ( 0 , 2 ) & … & y ( R - 1 , 2 ) \\ . & . & … & . & . & … & . \\ . & . & … & . & . & … & . \\ . & . & … & . & . & … & . \\ x ( 0 , P - 1 ) & x ( 1 , P - 1 ) & … & x ( Q - 1 , P - 1 ) & y ( 0 , P - 1 ) & … & y ( R - 1 , P - 1 )\end{matrix} ) \\ [ Math . 4 ]$$

$$( 4 )$$

$$( \begin{matrix}s ( 0 , t ) \\ s ( 1 , t ) \\ s ( 2 , t ) \\ . \\ . \\ . \\ s ( 18 , t ) \\ s ( 19 , t )\end{matrix} ) = ( \begin{matrix}x ( 0 , 0 ) & x ( 1 , 0 ) & x ( 2 , 0 ) & x ( 3 , 0 ) & x ( 4 , 0 ) & x ( 5 , 0 ) & y ( 0 , 0 ) & y ( 1 , 0 ) \\ x ( 0 , 1 ) & x ( 1 , 1 ) & x ( 2 , 1 ) & x ( 3 , 1 ) & x ( 4 , 1 ) & x ( 5 , 1 ) & y ( 0 , 1 ) & y ( 1 , 1 ) \\ x ( 0 , 2 ) & x ( 1 , 2 ) & x ( 2 , 2 ) & x ( 3 , 2 ) & x ( 4 , 2 ) & x ( 5 , 2 ) & y ( 0 , 2 ) & y ( 1 , 2 ) \\ . & . & . & . & . & . & . & . \\ . & . & . & . & . & . & . & . \\ . & . & . & . & . & . & . & . \\ x ( 0 , 18 ) & x ( 1 , 18 ) & x ( 2 , 18 ) & x ( 3 , 18 ) & x ( 4 , 18 ) & x ( 5 , 18 ) & y ( 0 , 18 ) & y ( 1 , 18 ) \\ x ( 0 , 19 ) & x ( 1 , 19 ) & x ( 2 , 19 ) & x ( 3 , 19 ) & x ( 4 , 19 ) & x ( 5 , 19 ) & y ( 0 , 19 ) & y ( 1 , 19 )\end{matrix} ) \\ ( \begin{matrix}f ( 0 , t ) \\ f ( 1 , t ) \\ f ( 2 , t ) \\ f ( 3 , t ) \\ f ( 4 , t ) \\ f ( 5 , t ) \\ n ( 0 , t ) \\ n ( 1 , t )\end{matrix} ) + ( \begin{matrix}b ( 0 , t ) \\ b ( 1 , t ) \\ b ( 2 , t ) \\ . \\ . \\ . \\ b ( 18 , t ) \\ b ( 19 , t )\end{matrix} )$$

The formula (3) is a matrix conversion method that regards a noise as a fluorescent substance, assumes that the sample contains Q type of a labeled fluorescent substance used in the sample and R type of a noise fluorescent substance, and converts fluorescence spectral intensities into fluorescent substance type intensities in Q+R types of fluorescent substances.

When the noise fluorescence is not detected, the matrix N≈0, and thus, an ordinary conversion is performed. When a noise peak is detected by electrophoresis, it is effective to obtain the matrix F and the matrix N in computing a base species and the like.

The matrix X and the matrix Y are fixed values determined by electrophoresis conditions, such as a fluorescent substance type and a fluorescence spectrum dividing condition. From these values and the measured matrix S and matrix B, the matrix F and the matrix N at each time are determined by the least squares method. With this process, the labeled fluorescent substance intensity waveform matrix F from which an effect of the noise fluorescence peak is removed can be obtained, and accurate values of a base species and a fragment type can be obtained (an analysis method 1).

Computing an intensity by each detection wavelength band, that is, a matrix YN from the matrix N obtained in the above-described calculation, and subtracting it from the matrix S ensure obtaining an electropherogram from which the noise peak is removed (an analysis method 2).

### <Analysis Process in Data Processing Unit>

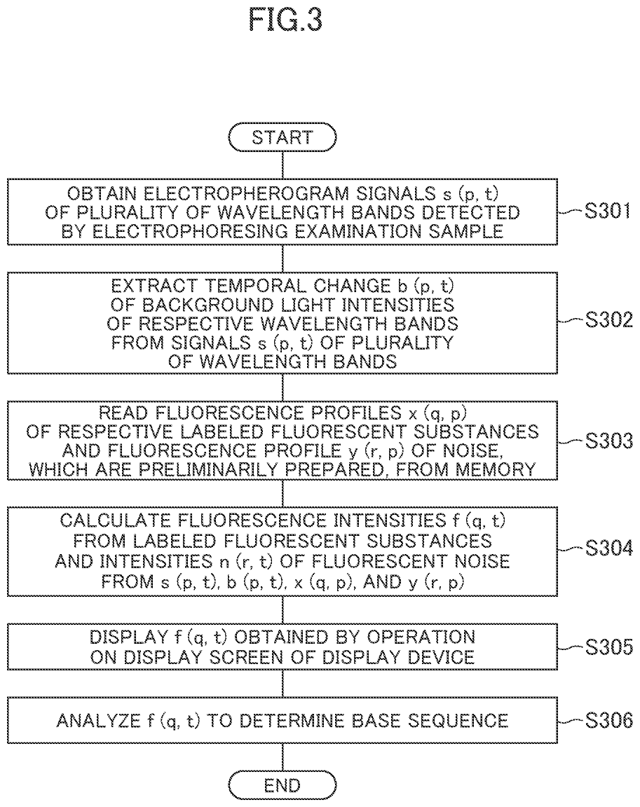

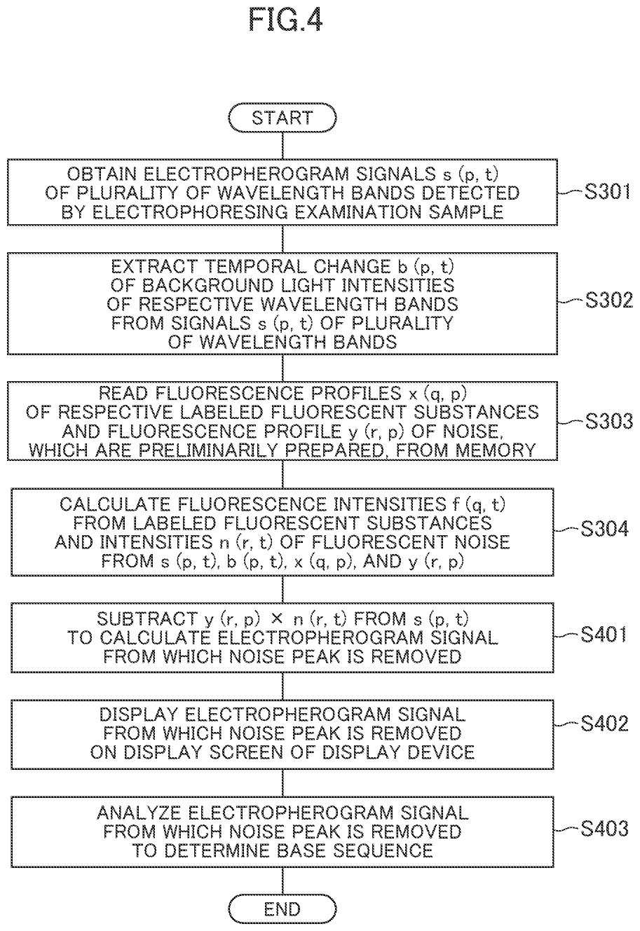

Here, the above-described analysis methods 1 and 2 are described as processes executed by the data processing unit 101. FIG. 3 is a flowchart for describing an electrophoresis data analyzing process executed by the data processing unit 101 based on the analysis method 1. FIG. 4 is a flowchart for describing an electrophoresis data analyzing process executed by the data processing unit 101 based on the analysis method 2.

(i) Process Based on Analysis Method 1

(i-1) Step 301

The detection mechanism unit 37 detects fluorescence generated from an examination sample by irradiating the examination sample with the laser light 9. In the detection mechanism unit 37, the two-dimensional detector 34 is divided into P from a detection wavelength band 0 to P−1 (P: the number of divided wavelengths, such as P=20), and detection data at a predetermined electrophoresis time t (for example, t=0 to 10000) is repeatedly output. The data processing unit 101 obtains the detection data repeatedly output from the detection mechanism unit 37 as an electropherogram signal (electrophoresis data) s (p, t). That is, electropherogram signals of the number of divided wavelength bands (P) can be obtained here. The data processing unit 101, for example, temporarily stores the electropherogram signals s (p, t) of the sequentially obtained respective wavelength bands in the memory 102.

(i-2) Step 302

The data processing unit 101 reads out the electropherogram signals in the respective wavelength bands from the memory 102, and extracts each signal that exhibits a non-pulsive fluctuation from the signals as a signal b (p, t) of a temporal change of the background intensity. That is, since the electropherogram signal is obtained for each divided wavelength band (P division), P temporal change of the background intensity is extracted. More specifically, a fluorescence intensity signal as a high-frequency component is removed by, for example, applying a low-pass filter on the electropherogram signal s (p, t), and moreover, troughs of the waveform are detected and a signal obtained by connecting the positions of the troughs can be set as the temporal change b (p, t) of the background intensity. Alternatively, there is, for example, a method to set a temporal change of the background intensity by obtaining the minimum intensity by each constant section and connecting them.

(i-3) Step 303

The data processing unit 101 reads fluorescence profiles of the respective labeled fluorescent substances used in the examination sample and a fluorescence profile of a fluorescent substance other than the labeled fluorescent substances (a non-labeled fluorescent substance: for example, a noise), which are preliminarily prepared, from the memory 102. The respective labeled fluorescent substance profiles are profiles uniquely specified when types of the labeled fluorescent substances are found. The fluorescence profile of a noise is determined by assuming a feature of a profile the noise has, and analyzing each piece of a plurality of the electrophoresis data (the electropherogram signals) obtained in the past based on the assumed profile feature. Accordingly, theses profiles are fixed values determined by electrophoresis conditions (a fluorescent substance type, a division condition, and the like). For example, each of the labeled fluorescent substance profiles and the noise fluorescence profile have been obtained before the electrophoresis is executed, and are stored in the memory 102 in advance.

(i-4) Step 304

The above-described formula (2) or (4) specifies a relation between the detected electropherogram signal s (p, t), the background intensity b (p, t) during the electrophoresis, the fluorescence profiles x (q, p) of the respective labeled fluorescent substance, and the set noise profile y (r, p); and the fluorescence intensity f (q, t) from the labeled fluorescent substance during the electrophoresis and the intensity n (r, t) of the fluorescent noise during the electrophoresis, in the predetermined number of divided wavelengths.

The data processing unit 101 calculates the fluorescence intensity f (q, t) at each time and the fluorescent noise intensity n (r, t) at each time using the least squares method (one example) based, for example, on the formula (4).

(i-5) Step 305

The data processing unit 101 displays the fluorescence intensity f (q, t) at each time calculated at Step 304 on the output device (a display device) 105 by each labeled fluorescent substance (for example, see FIG. 10 of Example 2).

(i-6) Step 306

The data processing unit 101 analyzes the fluorescence intensity f (q, t) at each time calculated at Step 304 to determine a base sequence included the examination sample. Information on the determined base sequence may be displayed on the output device (the display device) 105. Note that a well-known method (for example, the method described in Patent Literature 1) can be used for the determination method of the base sequence.

(ii) Process Based on Analysis Method 2

In the analysis method 2, the same processes as in the analysis method 1 are performed from Steps 301 to 304. Therefore, only Steps 401 to 403, which are different from the analysis method 1, are described here.

(ii-1) Step 401

The data processing unit 101 multiplies the noise fluorescence profile y (r, p) read from the memory 102 by the fluorescent noise intensity n (r, t) during the electrophoresis calculated at Step 304, and subtracts this from the detected electropherogram signal s (p, t) of each wavelength band to obtain the electropherogram signal from which a noise peak component is removed.

(ii-2) Step 402

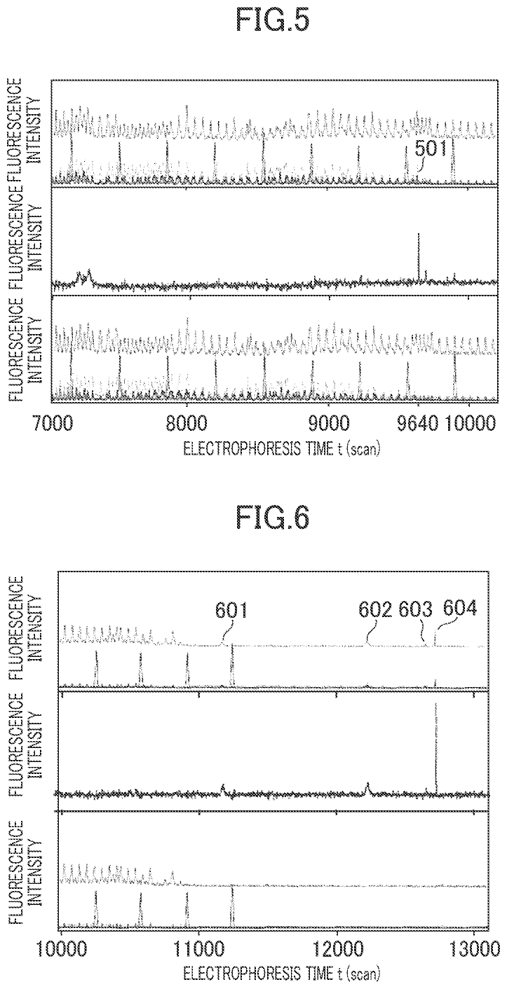

The data processing unit 101 displays the electropherogram signal of each wavelength band from which the noise peak component is removed, which is calculated at Step 401, on the output device (the display device) 105 (for example, see a lower part in FIG. 5 and a lower part in FIG. 6 of Example 1).

(ii-3) Step 403

The data processing unit 101 analyzes the electropherogram signal of each wavelength band from which the noise peak component is removed, which is calculated at Step 402, to determine a base sequence contained in the examination sample. Information on the determined base sequence may be displayed on the output device (the display device) 105. Note that a well-known method (for example, the method described in Patent Literature 1) can be used for the determination method of the base sequence.

### Example 1

FIG. 5 is a drawing illustrating an effect of a noise fluorescence removal according to Example 1. Example 1 is a measurement result obtained based on the analysis method 2. An upper part in FIG. 5 illustrates a temporal change of a measured (detected) electropherogram s (p, t), a middle part in FIG. 5 illustrates a temporal change of a noise fluorescence n (r, t) obtained by an operation, and the lower part in FIG. 5 illustrates an electropherogram with a less effect of a noise fluorescence peak, obtained by removing a detection wavelength band component based on the n (r, t) from the s (p, t).

FIG. 5 illustrates measurement and arithmetic operation results when fluorescent substance types were five types and when one type of a noise fluorescence had a fluorescence profile preliminarily measured and set. In FIG. 5, the temporal change of s (p, t) was illustrated by extracting 2nd, 5th, 8th, 11th, 14th, and 17th intensities of signals divided into twenty (each waveform indicates an intensity change of six detection wavelength bands). A noise peak 501 was detected near the electrophoresis time t=9640 scan. The noise peak 501 could be confirmed as a noise since a detected band width was smaller than a band width of a fluorescent substance fragment. Subtracting the detection wavelength band based on the noise fluorescence component (the signal waveform in the middle part in FIG. 5) from the temporal change of the signal waveforms s (p, t) in the upper part in FIG. 5 ensures obtaining the waveforms from which the noise peak is removed (the signal waveforms in the lower part in FIG. 5), thereby ensuring an accurate base analysis.

While it is not illustrated in FIG. 5, even when the temporal change of the fluorescence intensity f (q, t) from the labeled fluorescent substance is directly determined based on the formula (3) (the analysis method 1), the fluorescence intensity waveform from which the noise peak is removed can be obtained. Thus, even when a noise is overlaid on a peak of an electrophoresis band of a native fluorescent substance, its effect can be removed.

### Example 2

FIG. 6 is a drawing illustrating an effect of a noise fluorescence removal according to Example 2. Example 2 is a measurement result obtained based on the analysis method 2 similar to Example 1. In FIG. 6, similarly to FIG. 1, an upper part in FIG. 6 illustrates a temporal change of a measured (detected) electropherogram s (p, t), a middle part in FIG. 6 illustrates a temporal change of a noise fluorescence n (r, t) obtained by an operation, and the lower part in FIG. 6 illustrates an electropherogram with a less effect of a noise fluorescence peak, obtained by removing a detection wavelength band intensity component based on the noise fluorescence component from the s (p, t).

In Example 2, noise peaks are detected near the electrophoresis time t=11170, 12220, 12650, and 12720 scans, and noise peaks thereof 601 to 604 are seen in the s (p, t). The peaks 601 and 602 near 11170 and 12220 scans are found to be different from the labeled fluorescent substance by judging from the fluorescence profile. The peaks 603 and 604 near 12650 and 12720 scans are identified as noises also from narrowed band widths compared with many other electrophoresis bands. Thus, it was confirmed that the noise peak could be determined. Subtracting the detection wavelength band intensity component based on the computed noise fluorescence component (the signal waveform in the middle part in FIG. 6) from the temporal change of the s (p, t) in the upper part in FIG. 6 obtained the signal waveforms from which the noise peaks were removed (the signal waveforms in the lower part in FIG. 6). Based on these signal waveforms from which the noise peaks are removed, the base analysis can be accurately executed.

### Example 3

Example 3 illustrates an effect of a result based on the analysis method 1. Example 3 illustrates an example where four types of labeled fluorescent substances were used when a sample for determining a base sequence is measured. As fluorescent substances 1, 2, 3, and 4, respective fluorescent substances having the maximum wavelengths of fluorescence of 528 nm, 549 nm, 575 nm, and 607 nm are used. The two-dimensional detector 34 with the number of pixels in the X direction of 256 or 512 pixels is used, and a fluorescent image is formed by dispersing the wavelength to approximately 0.72 nm/pixel. The detection wavelength ranges are set to W1=520 m and W2=692 nm, and are detected by dividing them into twenty divisions of approximately equal wavelength band widths (widths of respective wavelength bands are approximately 8.6 nm). The two-dimensional detector 34 computes the intensity by integrating an intensity by approximately each 12 pixel.

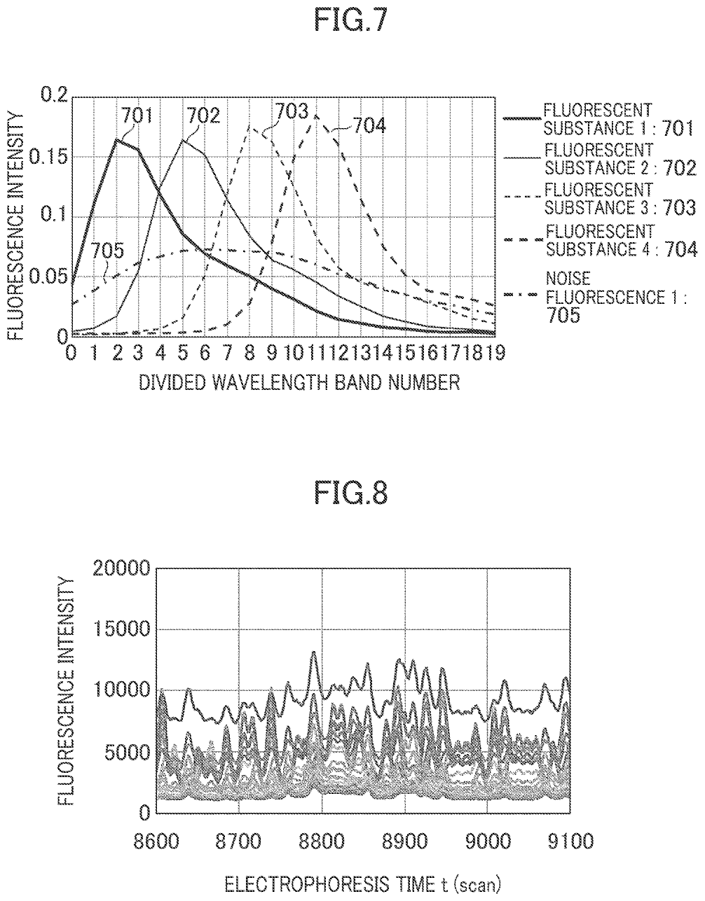

FIG. 7 illustrates fluorescence spectral profiles: x (q, p) of the labeled fluorescent substances used in Example 3 and a noise fluorescence profile: y (r, p) (q=0, 1, 2, 3, r=0, p=0, 1, 2, . . . , 19). In Example 3, the labeled fluorescent substances were four types of the fluorescent substances 1, 2, 3, and 4, and the noise fluorescent substance was one type, and the respective fluorescence intensity properties have been additionally analyzed in advance and their fluorescence profiles were obtained. FIG. 7 illustrates profiles 701 to 704 of the labeled fluorescent substances 1 to 4, and a profile 705 of a noise fluorescent substance 1. Note that it is illustrated that the signal intensity is normalized such that the accumulated values of the intensities in all the divided wavelength bands becomes 1.

As illustrated in FIG. 7, the four types of labeled fluorescent substances and one type of noise fluorescent substance have different wavelength profiles, and can be inversely converted by the least squares method from the above-described formula (3). Therefore, as described in FIG. 3, the data processing unit 101 operates the fluorescence intensity waveforms: f (0, t), f (1, t), f (2, t), and f (3, t) from the labeled fluorescent substances during the electrophoresis and the intensity waveform: n (0, t) of the noise fluorescent substance using the least squares method.

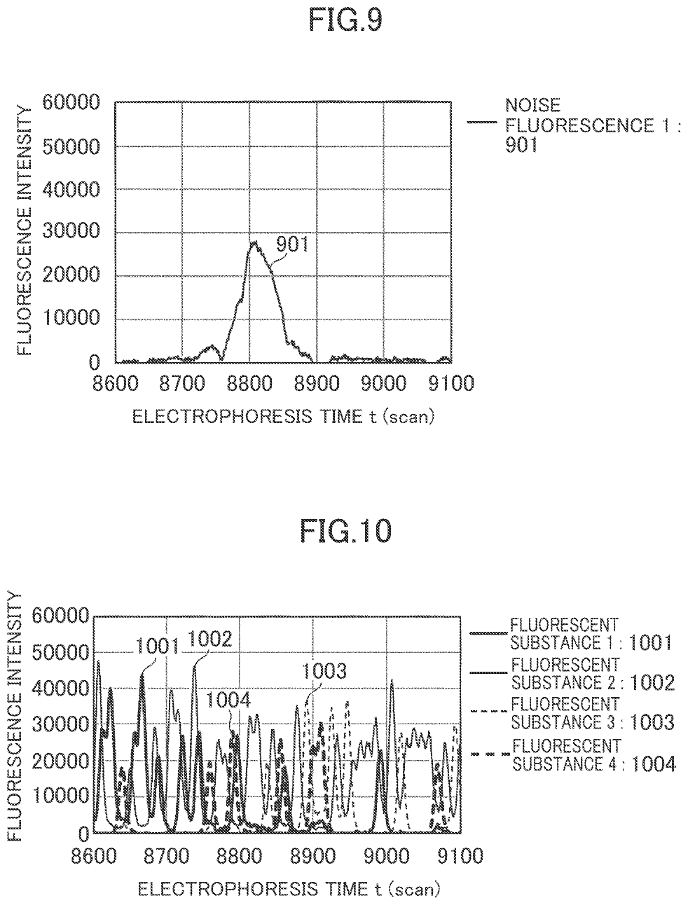

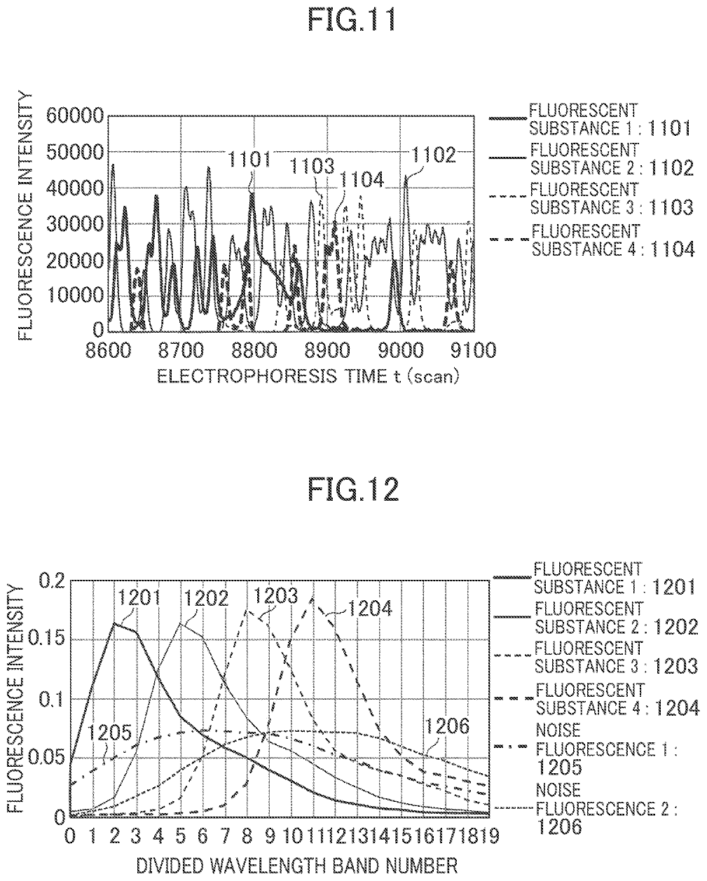

FIG. 8 illustrates an exemplary electropherogram s (p, t) during the electrophoresis measured. In FIG. 8, the electrophoresis time t=8600 scan to 9100 scan, and respective intensity changes of twenty wavelength bands from a detection wavelength 0 to a detection wavelength 19 are illustrated. FIG. 9 is a drawing illustrating a part of the intensity waveform: n (0, t) of the noise fluorescent substance (a signal intensity 901 of the noise fluorescence 1) analyzed by the least squares method. As seen from FIG. 9, a noise peak is detected near t=8800 scan. However, with the method of the disclosure (the analysis method 1), even when such a noise peak is detected, the fluorescence intensity waveform from the labeled fluorescent substance can be analyzed without the effect thereof. FIG. 10 illustrates a result of noise peak removal by the operation (the fluorescence intensity waveform f (0, t), f (1, t), f (2, t), and f (3, t) from the respective labeled fluorescent substances: intensity waveforms 1001 to 1004 of the fluorescent substances 1 to 4). Meanwhile, FIG. 11 illustrates the fluorescence intensity waveforms from the labeled fluorescent substances (intensity waveforms 1101 to 1104 from the fluorescent substances 1 to 4) calculated without setting the noise fluorescent substance as a comparative example. The fluorescence intensity waveform 1101 of the fluorescent substance 1 in FIG. 11 is blunt from the electrophoresis time t=8800 to 8850, and is seen to be affected by the fluorescence intensity waveform (FIG. 9) of the noise. Therefore, it is possible to misdetermine a base identification in the part with blunt waveform. That is, while the possibility of error decreases when the bluntness of the intensity waveform 1101 of the fluorescent substance 1 is small, it is also possible that the base of the fluorescent substance is overlapped and displayed when the bluntness of the intensity waveform 1101 is large. Accordingly, the noise component has to be removed in order to accurately identify the base. In contrast to this, it is seen that the fluorescence intensity from the labeled fluorescent substance near the noise peak (near the electrophoresis time t=8800) is more accurately computed in the fluorescence intensity waveform f (q, t): (q=0, 1, 2, 3) illustrated in FIG. 10.

Note that while one type of the noise fluorescent substance was set in the Example, it is possible to remove a noise with a different profile if two types are set, thereby enhancing more accuracy. For example, FIG. 12 is a drawing illustrating profiles of four types of labeled fluorescent substances (profiles 1201 to 1204 from the fluorescent substances 1 to 4) and profiles of two types of noise fluorescent substances (profiles 1205 and 1206 of the noise fluorescences 1 and 2). Also in this case, the four types of labeled fluorescent substances 1201 to 1204 and the two types of noise fluorescent substances 1205 and 1206 have wavelength profiles different from one another, and are possible to be identified.

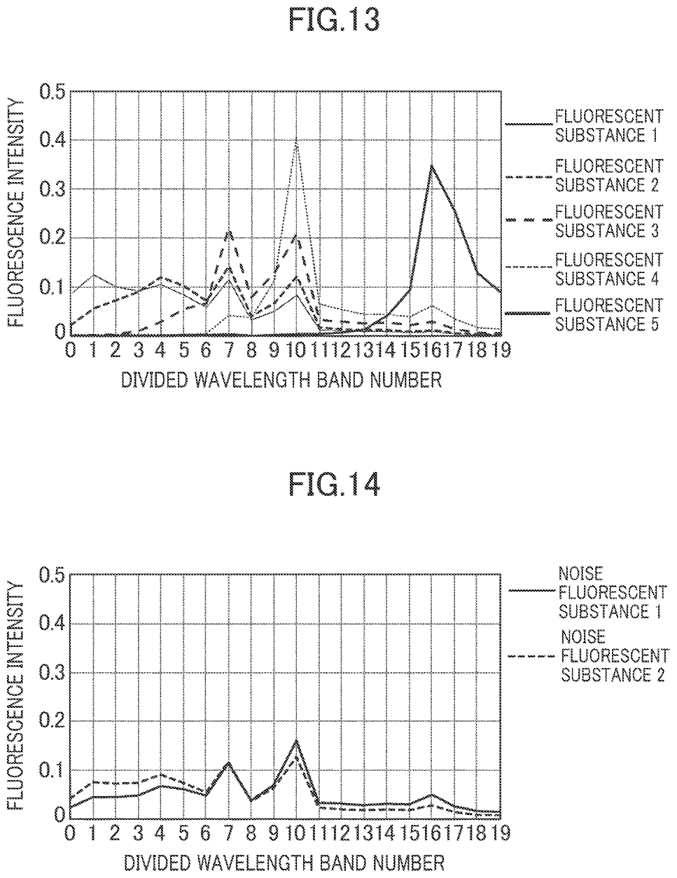

When the detection wavelength region is divided and detected, the detection wavelength bands do not necessarily have to be continuous, but discontinuous (discrete) wavelength bands may be used. Furthermore, instead of the same width for each wavelength band (the detection wavelength band width is equal: it is equally set in Example 3), the wavelength widths of the respective wavelength bands may be any widths (for example: unequally set detection wavelength band width: a wavelength band width of a peak part is set larger than the other parts in Example 4 (FIGS. 13 and 14) described later). For example, it is possible to expand (increase) the width near the fluorescence maximum wavelength, contract (decrease) the width of the wavelength band where Raman scattering of the laser light 9 is detected, and not to detect the signal from the wavelength band. Since the effect of the Raman scattering of the laser light 9 that is not derived from the labeled fluorescent substance or the noise fluorescent substance appears in the detection signal when the detection wavelength width is continuous and equal, unequally setting the detection wavelength width is effective. The number of divisions does not have to be twenty as indicated in each Example. It is only necessary to set the fluorescence profile of the labeled fluorescent substance and the fluorescence profile of the noise fluorescent substance in those conditions.

### Example 4

In Example 4, five types of labeled fluorescent substances were used and five types of fragments were analyzed. As the labeled fluorescent substances 1, 2, 3, 4, and 5, respective fluorescent substances with the maximum wavelengths of fluorescence of approximately 520 nm, 550 nm, 570 nm, 590 nm, and 655 nm were used. The two-dimensional detector 34 with the number of pixels in the X direction of 256 or 512 pixels is used, and a fluorescent image is formed by dispersing the wavelength to approximately 0.72 nm/pixel. The detection wavelength ranges are set to W1=522.5 nm and W2=690 nm. The number of divided wavelength bands are twenty divisions, similar to Example 3. The wavelength widths (=the number of pixels) of the respective detection wavelength bands are not identical, but the wavelengths near the fluorescence maximum wavelength were set expanded (increased) and the other wavelengths were set contracted (decreased). The widths and the intervals of the detection wavelength bands were set random.

FIG. 13 is a drawing illustrating the fluorescence spectral profiles: x (q, p) of the labeled fluorescent substances used in Example 4. FIG. 14 is a drawing illustrating the noise fluorescence profiles: y (r, p) (q=0, 1, 2, 3, 4, r=0, 1, p=0, 1, 2, . . . , 19) used in Example 4. In Example 4, each of the fluorescence strength properties were additionally analyzed in advance using five types of the fluorescence 1, 2, 3, 4, and 5 for the labeled fluorescent substance and two types for the noise fluorescent substance, and their fluorescence profiles were obtained. The intensities of fluorescence profiles are displayed by standardizing the integrated value of the intensities of the wavelength bands becomes 1. Note that detection wavelength band numbers 1, 4, 7, 10, and 16 are set to detect fluorescence in approximately five fluorescent maximum wavelength ranges. As can be seen from FIG. 13 and FIG. 14, the five types of labeled fluorescent substances and the two types of noise fluorescent substances have different fluorescence profile from one another. Accordingly, the inverse conversion is possible using the least squares method based on formula (3). In view of this, the data processing unit 101 calculates the fluorescence intensity waveforms from the labeled fluorescent substances during the electrophoresis: f (q, t), and the intensity waveforms of the noise fluorescent substances: n (r, t) in accordance with the above-described analysis method 1.

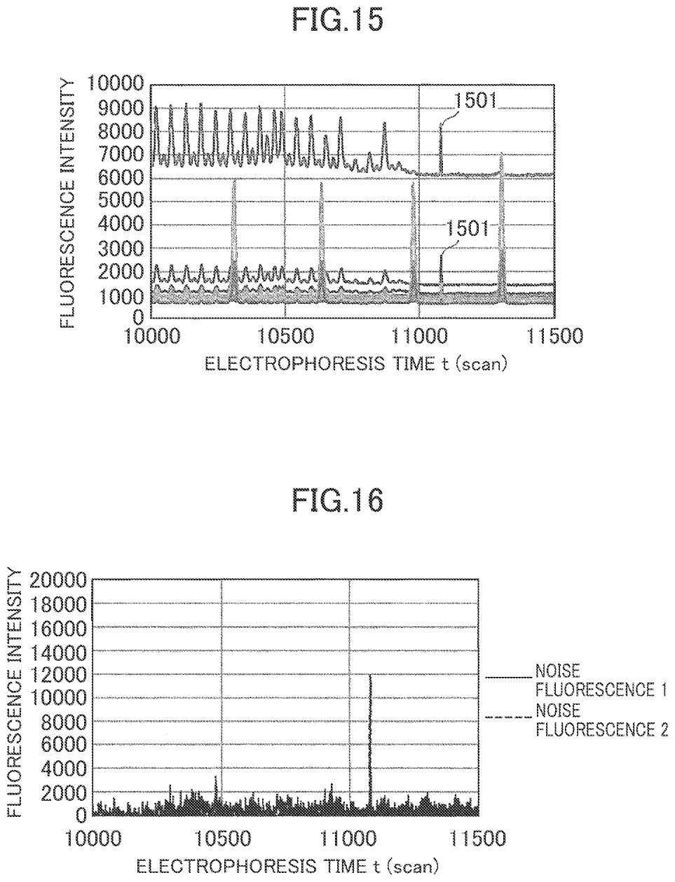

FIG. 15 is a drawing illustrating an exemplary electropherogram s (p, t) during the electrophoresis measured. FIG. 15 illustrates intensity changes of twenty wavelength bands from the detection wavelength band 0 to the detection wavelength band 19 from the electrophoresis time t=10000 scan to 115000 scan. FIG. 16 is a drawing illustrating parts of intensity waveforms of noise fluorescent substances: n (r, t) calculated in accordance with the analysis method 1. With reference to FIG. 15, a peak 1501 that is not fluorescence from the labeled fluorescent substances is detected near t=11100 scan. In FIG. 15, the fluorescence profile of the peak 1501 is different from the five types of labeled fluorescent substances, and the band width is apparently narrow compared with the fragments labeled with the fluorescent substances. In view of this, the peak 1501 can be recognized as a noise peak. However, in Example 4, the fluorescence intensity waveform from the labeled fluorescent substance could be analyzed without the effect thereof even when such a noise peak 1501 was detected.

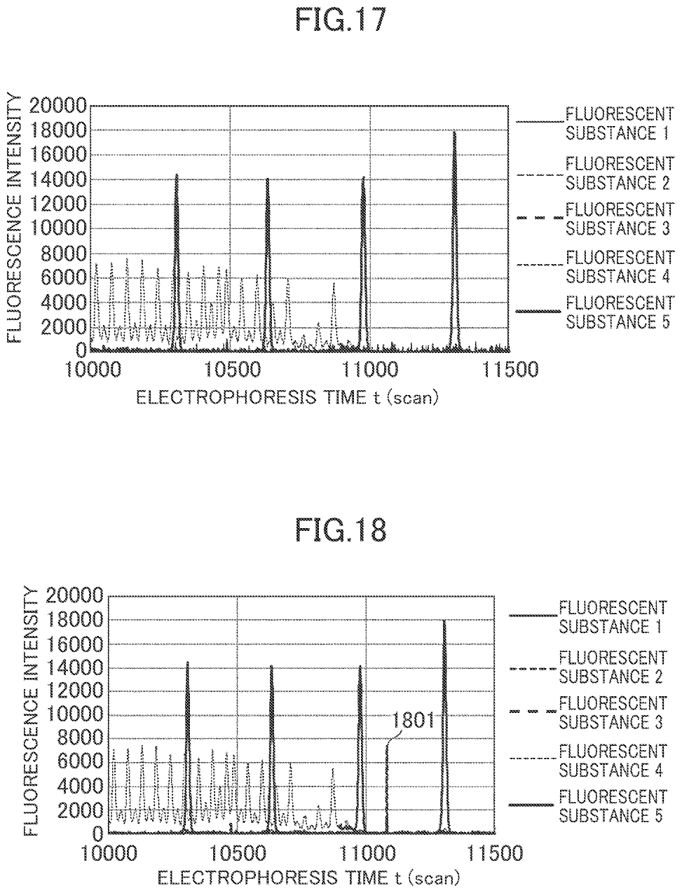

FIG. 17 is a drawing illustrating a result (the fluorescence intensity waveforms: f (q, t) from the labeled fluorescent substances during the electrophoresis) obtained by the operation of the analysis method 1. Meanwhile, FIG. 18 is a drawing illustrating the fluorescence intensity waveforms from the labeled fluorescent substances calculated without setting the fluorescence profile y (r, p) of the noise fluorescent substance as the comparative example. In FIG. 18, a sharp peak 1801 is recognized near t=11100 scan. In contrast to this, in the fluorescence intensity waveforms f (q, t) illustrated in FIG. 17, the peak 1801 does not appear. Then, the data processing unit 101 performs an analysis process on the fluorescence intensity waveforms from which the noise peak 1801 is removed. This ensures performing a fragment analysis with less noise effect.

Note that, in Example 4, the effect of the noise removal could be found even when one type of the noise fluorescent substance was set. The fragment analysis can accommodate various kinds of combinations of fluorescent substances, such as with the case of six or four types of labeled fluorescent substance types, and can identify the noise peak to reduce the effect thereof.

### <Reliability Display Processing of Electrophoresis Result>

In the above-described Examples 1 to 4, the fluorescence intensity from a substance other than the labeled fluorescent substance is extracted as the noise (see FIGS. 5, 6, 9, and 16). It is ideal that such a noise does not appear in an electrophoresis result, but it is very difficult to make it zero. Even though the mixture of the noise is inevitable, when the extracted noise appearance is frequent or the intensity (level) of the noise is too large, the reliability of the corresponding electrophoresis result (detection data) itself can be determined as low. Therefore, for example, a threshold of the noise appearance frequency and a threshold of the intensity that can be determined as low reliability are preset. Then, the data processing unit 101 determines whether the extracted noise appearance frequency and intensity exceed the above-described thresholds or not, determines that the reliability of the electrophoresis result is low when at least one threshold is exceeded, and outputs the determination result to the output device 105. The output format may be an alarm or an alert display may be displayed on the screen. By doing this, the electrophoretic apparatus that includes an electrophoresis evaluation determining unit evaluating the electrophoresis result from the peak intensity and its occurrence frequency can be provided. Then, an operator can determine whether the measurement of the electrophoresis should be executed again.

### <Summary>

(i) While in the embodiment, the sample is electrophoresed by capillary electrophoresis and the time waveform thereof is analyzed, the disclosure is not limited to the capillary electrophoresis, and is applicable generally to electrophoresis and has a similar effect. The emission of light by a substance other than the labeled fluorescent substance can be generated even when a measurement method other than the electrophoresis is used.

In the electrophoresis, when a reacted sample is electrophoresed in a medium having a molecular sieve effect (for example, a polymer aqueous solution), molecules flow in the order of the smallest molecular weight, and, in particular, in the case of DNA, each one base separates. Therefore, sequentially reading this ensures measuring the signal intensity. Since reading each one base is a basic sequence, other methods other than the electrophoresis can be used as a method for reading each one base. For example, the signal can be read by each one base by repeating a procedure, such as attaching a fluorescent substance by each one base on a substrate and reading them, then, removing them and attaching a fluorescent substance on the next base and reading it. Even in such a method and a device that detect the base sequence of DNA by sequentially reacting them, the fluorescence from other than the labeled fluorescent substance is overlaid in detecting reaction in some cases. That is, there is a case where the signal by the fluorescent substance other than the labeled fluorescent substance (regarded as the noise fluorescent substance) is detected, and this is the noise. Thus, even in the case where each one base is read, since the time information in the detected signal is basically the same as base electrophoresis that continuously reads the base, the noise is overlaid on the fluorescence intensity signal derived from the base and is detected. Then, identifying the emission of light by the substance other than the labeled fluorescent substance, setting its fluorescence profile, and performing a conversion operation assuming that the fluorescence from the labeled fluorescent substance and the fluorescent substance other than that emits lights ensure separating the intensity of the labeled fluorescent substance from the fluorescence intensity other than the labeled fluorescent substance, thereby ensuring more accurate computation of the base species.

Accordingly, applying the technique of the disclosure ensures removing the noise in a method other than the electrophoresis, similarly to the case of the electrophoresis.

(ii) While the embodiment has been described using DNA as an example, the technique of the disclosure is applicable to the biopolymer, such as polysaccharides, proteins (enzyme, peptide), and nucleic acids (DNA, RNA).

(iii) In the embodiment, the profile of Q type (Q is an integer of one or more) of labeled fluorescent substance used in the biopolymer and the profile of non-labeled fluorescent substance (for example, a noise) as R type (R is an integer of one or more) of fluorescent substance different from the labeled fluorescent substance are preset, and held in the memory 102 and the storage device 103. Using a measurement method, such as the electrophoresis, the temporal change of intensities of the plurality of wavelength bands are detected. Then, the data processing unit (for example, the processor) 101 reads the profile of the labeled fluorescent substance and the profile of the non-labeled fluorescent substance from the memory 102 and the like, and identify Q+R types of the fluorescent substances using the temporal change of the intensities of the plurality of wavelength bands, the profile of the Q type of labeled fluorescent substance, and the profile of the R type of non-labeled fluorescent substance. Furthermore, the data processing unit 101 analyzes the biopolymer from the data of the identified Q type of fluorescent substance. The analysis is executed using a well-known technique. Thus, introducing the profile of non-labeled fluorescent substance ensures computing the intensity of the labeled fluorescent substance itself without the effect of the noise caused by impurities, thereby ensuring accurately detection and identification of the component of the biopolymer.

In the embodiment, the detection wavelength range of a predetermined width (for example, 520 nm to 700 nm) is set, the detection wavelength range is divided into P (P is a positive integer: for example, twenty divisions) wavelength bands, and the temporal change (s (p, t)) of the intensities of the plurality of fluorescent substances are detected. By doing this, since the fluorescence intensity ratios of the respective labeled fluorescent substance are different by each of the wavelength bands, the labeled fluorescent substance and the non-labeled fluorescent substance can be accurately and efficiently detected and separated.

Specifically, the embodiment can identify the labeled fluorescent substance and the non-labeled fluorescent substance in two methods. One is the method that operates the f (q, t) from, for example, the above-described formula (1) (or formula (3)), and identify the Q type of fluorescent substance using the obtained f (q, t) (the analysis method 1). The other is the method that operates the n (r, t) from the formula (1), removes the fluorescence intensity of the non-labeled fluorescent substance by subtracting the detection wavelength band component based on the n (r, t) from the s (p, t), and identifies the Q type of fluorescent substance using the temporal change of the intensities of the plurality of fluorescent substances from which the non-labeled fluorescent substance is removed (the analysis method 2).

Furthermore, in the embodiment, the degree of reliability of the measurement result may be further evaluated by determining whether at least one of the appearance frequency of the R type of non-labeled fluorescent substance and the intensity of the non-labeled fluorescent substance is equal to or more than the preset thresholds or not. By doing this, an operator can determine whether the measurement should be executed again.

(iv) The disclosure is not limited to the above-described embodiment and Examples, but includes various modifications. The descriptions of the embodiment and Examples are described in detail for describing the technique of the disclosure clearly, and are not necessarily for limiting to include all the described configurations. It is possible to replace a part of configuration of a certain embodiment with a configuration of another embodiment, and it is also possible to add a configuration of another embodiment to a configuration of a certain embodiment. Another configuration can be added to, deleted from, and replaced with a part of configuration of each Example.

Besides four to six types, various types of the labeled fluorescent substances are applicable. It is also possible to set a plurality of types of noise fluorescent substances, besides one type and two types. For the combinations of fluorescent substances, various kinds of combinations are possible besides the Example description. For the setting of the detection wavelength band, the number of divisions can be made more. Setting the respective fluorescence profiles corresponding to these combinations ensures a similar analysis. Furthermore, while in the above-described Example, DNA is the measurement target, it is applicable to a method and an apparatus for separately detecting a biologically-relevant element, such as protein, and the measurement that is insusceptible to the fluorescent component derived from impurities or the measurement with less effect can be performed similarly.

### LIST OF REFERENCE SIGNS

1 capillary array
2 negative electrode
3 buffer liquid on negative electrode side
4 gel block
5 gel block coupling portion
6 valve
7 earth electrode
8 light irradiation position
9 laser light
10 syringe
11 oven
12 buffer liquid on earth electrode side
15 array platform
16 capillary
20 light source
21 high-voltage power supply
22 sample introduction portion
23 first buffer container
24 flow medium injection mechanism
25 second buffer container
26 detector
31 fluorescence condenser lens
32 grating
33 focus lens
34 two-dimensional detector
35 light emission from capillary portion
36 light flux made of light emission from capillary portion parallelized by fluorescence condenser lens
37 detection mechanism unit of fluorescence
100 capillary electrophoretic apparatus
101 data processing unit
102 memory
103 storage device
104 input device
105 output device

1. A biopolymer analysis method for analyzing a biopolymer by using the biopolymer as a sample and a plurality of types of fluorescent substances as labels and detecting respective fluorescence intensities, the biopolymer analysis method comprising:
setting a profile of Q type (Q is an integer of one or more) of labeled fluorescent substance used in the sample;
setting a profile of a non-labeled fluorescent substance as R type (R is an integer of one or more) of fluorescent substance different from the labeled fluorescent substance;
detecting a fluorescence intensity from the sample using a predetermined measurement method; and
identifying Q+R types of fluorescent substances using the fluorescence intensity, the profile of the Q type of labeled fluorescent substance, and the profile of the R type of non-labeled fluorescent substance.

2. The biopolymer analysis method according to claim 1, further comprising
analyzing the biopolymer from data of the identified Q type of fluorescent substance.

3. The biopolymer analysis method according to claim 1,
wherein in the detecting of the fluorescence intensity from the sample, a detection wavelength range of a predetermined width is set, and the detection wavelength range is divided into P (P is a positive integer) wavelength bands and detected.

4. The biopolymer analysis method according to claim 3,
wherein when a detection intensity per divided wavelength band is s (p, t), the profile of the Q type of labeled fluorescent substance is x (q, p), the profile of the R type of non-labeled fluorescent substance is y (r, p), a background intensity during a measurement is b (p, t), a fluorescence intensity from the labeled fluorescent substance is f (q, t), and a fluorescence intensity from the non-labeled fluorescent substance is n (r, t), the Q+R types of fluorescent substances are identified from the following formula.
$$( \begin{matrix}s ( 0 , t ) \\ s ( 1 , t ) \\ s ( 2 , t ) \\ . \\ . \\ . \\ s ( P - 1 , t )\end{matrix} ) = ( \begin{matrix}x ( 0 , 0 ) & x ( 1 , 0 ) & … & x ( Q - 1 , 0 ) \\ x ( 0 , 1 ) & x ( 1 , 1 ) & … & x ( Q - 1 , 1 ) \\ x ( 0 , 2 ) & x ( 1 , 2 ) & … & x ( Q - 1 , 2 ) \\ . & . & … & . \\ . & . & … & . \\ . & . & … & . \\ x ( 0 , P - 1 ) & x ( 1 , P - 1 ) & … & x ( Q - 1 , P - 1 )\end{matrix} ) ( \begin{matrix}f ( 0 , t ) \\ f ( 1 , t ) \\ . \\ . \\ . \\ f ( Q - 1 , t )\end{matrix} ) + ( \begin{matrix}y ( 0 , 0 ) & … & r ( R - 1 , 0 ) \\ y ( 0 , 1 ) & … & r ( R - 1 , 1 ) \\ y ( 0 , 2 ) & … & r ( R - 1 , 2 ) \\ . & … & . \\ . & … & . \\ . & … & . \\ y ( 0 , P - 1 ) & … & r ( R - 1 , P - 1 )\end{matrix} ) ( \begin{matrix}n ( 0 , t ) \\ . \\ . \\ . \\ n ( R - 1 , t )\end{matrix} ) + ( \begin{matrix}b ( 0 , t ) \\ b ( 1 , t ) \\ b ( 2 , t ) \\ . \\ . \\ . \\ b ( P - 1 , t )\end{matrix} ) or$$
$$( \begin{matrix}s ( 0 , t ) \\ s ( 1 , t ) \\ s ( 2 , t ) \\ . \\ . \\ . \\ s ( P - 1 , t )\end{matrix} ) = ( \begin{matrix}x ( 0 , 0 ) & x ( 1 , 0 ) & … & x ( Q - 1 , 0 ) & y ( 0 , 0 ) & … & r ( R - 1 , 0 ) \\ x ( 0 , 1 ) & x ( 1 , 1 ) & … & x ( Q - 1 , 1 ) & y ( 0 , 1 ) & … & r ( R - 1 , 1 ) \\ x ( 0 , 2 ) & x ( 1 , 2 ) & … & x ( Q - 1 , 2 ) & y ( 0 , 2 ) & … & r ( R - 1 , 2 ) \\ . & . & … & . & . & … & . \\ . & . & … & . & . & … & . \\ . & . & … & . & . & … & . \\ x ( 0 , P - 1 ) & x ( 1 , P - 1 ) & … & x ( Q - 1 , P - 1 ) & y ( 0 , P - 1 ) & … & r ( R - 1 , P - 1 )\end{matrix} ) ( \begin{matrix}f ( 0 , t ) \\ f ( 1 , t ) \\ . \\ . \\ . \\ f ( Q - 1 , t ) \\ n ( 0 , t ) \\ . \\ . \\ . \\ n ( R - 1 , t )\end{matrix} ) + ( \begin{matrix}b ( 0 , t ) \\ b ( 1 , t ) \\ b ( 2 , t ) \\ . \\ . \\ . \\ b ( P - 1 , t )\end{matrix} )$$
Here, t is time,
p is a number of the divided wavelength band (p=0, 1, . . . , P−1),
q is a number of a labeled fluorescent substance type (q=0, 1, . . . , Q−1), and
r is a number of the non-labeled fluorescent substance (r=0, 1, . . . , R−1).

5. The biopolymer analysis method according to claim 4,
wherein f (q, t) is computed by the formula, and the Q type of fluorescent substance is identified.

6. The biopolymer analysis method according to claim 4, comprising:
computing n (r, t) by the formula and subtracting a signal intensity caused by the n (r, t) from the s (p, t) to compute a detection intensity per divided wavelength band from which the non-labeled fluorescent substance is removed; and
identifying the Q type of fluorescent substance.

7. The biopolymer analysis method according to claim 1, comprising
electrophoresing the sample in a capillary or sequentially reacting the sample.

8. The biopolymer analysis method according to claim 1, further comprising
evaluating a degree of reliability of a measurement result by the predetermined measurement method by determining whether at least one of an appearance frequency of the R type of non-labeled fluorescent substance and an intensity of the non-labeled fluorescent substance is equal to or more than a preliminarily set threshold or not.

9. A biopolymer analyzer for analyzing a biopolymer by using the biopolymer as a sample and a plurality of types of fluorescent substances as labels and detecting respective fluorescence intensities, the biopolymer analyzer comprising:
a measurement unit that detects the fluorescence intensity from the sample using a predetermined measurement method;
a memory that stores a profile of Q type (Q is an integer of one or more) of labeled fluorescent substance used in the sample and a profile of a non-labeled fluorescent substance as R type (R is an integer of one or more) of fluorescent substance different from the labeled fluorescent substance; and
a data processing unit that reads the profile of the Q type of labeled fluorescent substance and the profile of the R type of non-labeled fluorescent substance from the memory, and identifies Q+R types of fluorescent substances using the detection intensity, the profile of the Q type of labeled fluorescent substance, and the profile of the R type of non-labeled fluorescent substance.

10. The biopolymer analyzer according to claim 9,
wherein the data processing unit further analyzes the biopolymer from data of the identified Q type of fluorescent substance.

11. The biopolymer analyzer according to claim 9,
wherein the measurement unit divides a preset detection wavelength range of a predetermined width into P (P is a positive integer) wavelength bands and detects.

12. The biopolymer analyzer according to claim 11,
wherein when a detection intensity per divided wavelength band is s (p, t), the profile of the Q type of labeled fluorescent substance is x (q, p), the profile of the R type of non-labeled fluorescent substance is y (r, p), a background intensity during a measurement is b (p, t), a fluorescence intensity from the labeled fluorescent substance is f (q, t), and a fluorescence intensity from the non-labeled fluorescent substance is n (r, t), the data processing unit identifies the Q+R types of fluorescent substances from the following formula.
$$( \begin{matrix}s ( 0 , t ) \\ s ( 1 , t ) \\ s ( 2 , t ) \\ . \\ . \\ . \\ s ( P - 1 , t )\end{matrix} ) = ( \begin{matrix}x ( 0 , 0 ) & x ( 1 , 0 ) & … & x ( Q - 1 , 0 ) \\ x ( 0 , 1 ) & x ( 1 , 1 ) & … & x ( Q - 1 , 1 ) \\ x ( 0 , 2 ) & x ( 1 , 2 ) & … & x ( Q - 1 , 2 ) \\ . & . & … & . \\ . & . & … & . \\ . & . & … & . \\ x ( 0 , P - 1 ) & x ( 1 , P - 1 ) & … & x ( Q - 1 , P - 1 )\end{matrix} ) ( \begin{matrix}f ( 0 , t ) \\ f ( 1 , t ) \\ . \\ . \\ . \\ f ( Q - 1 , t )\end{matrix} ) + ( \begin{matrix}y ( 0 , 0 ) & … & r ( R - 1 , 0 ) \\ y ( 0 , 1 ) & … & r ( R - 1 , 1 ) \\ y ( 0 , 2 ) & … & r ( R - 1 , 2 ) \\ . & … & . \\ . & … & . \\ . & … & . \\ y ( 0 , P - 1 ) & … & r ( R - 1 , P - 1 )\end{matrix} ) ( \begin{matrix}n ( 0 , t ) \\ . \\ . \\ . \\ n ( R - 1 , t )\end{matrix} ) + ( \begin{matrix}b ( 0 , t ) \\ b ( 1 , t ) \\ b ( 2 , t ) \\ . \\ . \\ . \\ b ( P - 1 , t )\end{matrix} ) or$$
$$( \begin{matrix}s ( 0 , t ) \\ s ( 1 , t ) \\ s ( 2 , t ) \\ . \\ . \\ . \\ s ( P - 1 , t )\end{matrix} ) = ( \begin{matrix}x ( 0 , 0 ) & x ( 1 , 0 ) & … & x ( Q - 1 , 0 ) & y ( 0 , 0 ) & … & r ( R - 1 , 0 ) \\ x ( 0 , 1 ) & x ( 1 , 1 ) & … & x ( Q - 1 , 1 ) & y ( 0 , 1 ) & … & r ( R - 1 , 1 ) \\ x ( 0 , 2 ) & x ( 1 , 2 ) & … & x ( Q - 1 , 2 ) & y ( 0 , 2 ) & … & r ( R - 1 , 2 ) \\ . & . & … & . & . & … & . \\ . & . & … & . & . & … & . \\ . & . & … & . & . & … & . \\ x ( 0 , P - 1 ) & x ( 1 , P - 1 ) & … & x ( Q - 1 , P - 1 ) & y ( 0 , P - 1 ) & … & r ( R - 1 , P - 1 )\end{matrix} ) ( \begin{matrix}f ( 0 , t ) \\ f ( 1 , t ) \\ . \\ . \\ . \\ f ( Q - 1 , t ) \\ n ( 0 , t ) \\ . \\ . \\ . \\ n ( R - 1 , t )\end{matrix} ) + ( \begin{matrix}b ( 0 , t ) \\ b ( 1 , t ) \\ b ( 2 , t ) \\ . \\ . \\ . \\ b ( P - 1 , t )\end{matrix} )$$
Here, t is time,
p is a number of the divided wavelength band (p=0, 1, . . . , P−1),
q is a number of a labeled fluorescent substance type (q=0, 1, . . . , Q−1), and
r is a number of the non-labeled fluorescent substance (r=0, 1, . . . , R−1).

13. The biopolymer analyzer according to claim 12,
wherein the data processing unit calculates f (q, t) by the formula, and identifies the Q type of fluorescent substance.

14. The biopolymer analyzer according to claim 12,
wherein by computing n (r, t) by the formula and subtracting a signal intensity caused by the n (r, t) from the s (p, t), the data processing unit has a function to compute a detection intensity per divided wavelength band from which the non-labeled fluorescent substance is removed and to display the intensity.

15. The biopolymer analyzer according to claim 9,
wherein the data processing unit further has a function to evaluate a degree of reliability of a measurement result by the predetermined measurement method by determining whether at least one of an appearance frequency of the R type of non-labeled fluorescent substance and an intensity of the non-labeled fluorescent substance is equal to or more than a preliminarily set threshold or not.

16. The biopolymer analyzer according to claim 9, further comprising
an electrophoresis mechanism unit that electrophoreses the sample or a sequential reaction mechanism unit that sequentially reacts the sample.

17. A biopolymer analysis method for analyzing a base sequence and/or a fragment type by labeling a biopolymer sample as DNA and/or oligonucleotide with a different fluorescent substance per base species or analysis fragment, and detecting fluorescence from the sample, the biopolymer analysis method comprising
setting a fluorescence profile of Q type of labeled fluorescent substance used in the sample and R type (R is one or more) of fluorescence profile that has a different fluorescence profile from the fluorescence profile of the labeled fluorescent substance, and identifying the Q type of fluorescent substance from the detected fluorescence intensity and the Q+R types of the fluorescence profiles.

18. A biopolymer analyzer for analyzing a base sequence and/or a fragment type by labeling a biopolymer sample as DNA and/or oligonucleotide with a different fluorescent substance per base species or analysis fragment and detecting fluorescence from the sample, the biopolymer analyzer Comprising
a data processing unit that identifies Q type of fluorescent substance from a fluorescence profile of the Q type of labeled fluorescent substance used in the sample, R type (R is one or more) of fluorescence profile that has a different fluorescence profile from the fluorescence profile of the labeled fluorescent substance, and the detected fluorescence intensity.
